(19) United States
(12) Patent Application Publication
(10) Pub. No.: US 2016/0106798 A1
(43) Pub. Date: Apr. 21, 2016

(54) TOPICAL TREATMENT FOR USE IN CONJUNCTION WITH ACUPUNCTURE OR ACUPRESSURE THERAPY

(71) Applicant: Lou Paradise, Rhinebeck, NY (US)

(72) Inventor: Lou Paradise, Rhinebeck, NY (US)

(21) Appl. No.: 14/884,383

(22) Filed: Oct. 15, 2015

Related U.S. Application Data
(60) Provisional application No. 62/063,976, filed on Oct. 15, 2014.

(51) Int. Cl. A61K 36/9068; A61K 9/06; A61K 36/28; A61K 36/22; A61K 36/77; A61K 47/12; A61K 36/53; A61K 36/49; A61K 31/455; A61K 45/06; A61K 35/583; A61K 47/10; A61K 9/00; A61K 36/81

(52) U.S. Cl. CPC A61K 36/9068; A61K 9/0014; A61K 9/06; A61K 36/28; A61K 36/22; A61K 36/77; A61K 36/81; A61K 36/53; A61K 36/49; A61K 31/455; A61K 45/06; A61K 35/583; A61K 47/10; A61K 47/12

(57) ABSTRACT

The present invention relates to a topical application of a homeopathic composition to the skin to be used as an enhancement for acupuncture and acupressure. The composition may be in the form of an ointment, cream, gel, or salve. The composition may be applied before, during, and after acupressure or acupuncture. The composition may contain snake venom along with a synergistic blend of vasodilator and analgesic herbs that relieve pain by stimulating blood flow and reducing edema. The composition complements the Chinese medicine theory of balancing the body's energy flow.

### CROSS REFERENCE TO RELATED APPLICATION

This application claims priority to the U.S. Provisional Patent Application Ser. No. 62/063,976, filed on Oct. 15, 2014 and entitled TOPICAL TREATMENT FOR USE IN CONJUNCTION WITH ACUPUNCTURE OR ACUPRESSURE THERAPY, the contents of which are relied upon and incorporated herein by reference.

### FIELD OF INVENTION

The present invention relates to methods and apparatus for combining a topical homeopathic ointment or cream and Chinese medicine methods. More particularly, the present invention relates to combining the topical application of a homeopathic composition and acupuncture and acupressure.

### BACKGROUND

Acupuncture is the Chinese medicine practice of inserting thin needles into specific points on the skin in order to balance the body's flow of energy by opening energy channels and promoting circulation. Acupressure is similar with the exception of applying pressure to specific pressure points. The specific pressure points are often not on the same part of the body as the pain. Acupuncture and acupressure have been used to treat musculoskeletal and neural pain, and sessions can last from 10 to 60 minutes. The needle or pressure applied increases blood flow at the localized pressure point on the skin due to vasodilation or opening of the body's energy channels. This in turn causes an increased flow of oxygen to the muscles, a release of tension due to nerve stimulation, and results in pain relief Pain relief can also be achieved by the needles stimulating nerves to signal to the central nervous system to release endorphins to dull the feeling of pain. Acupuncture or acupressure on its own likely does not reduce swelling at the site of the wound or pain.

To enhance vasodilation and pain relief, acupuncture has been coupled with the simultaneous use of heat lamps over the body, electro stimulation at the needle, or moxibustion over the needles. However, these three enhancements likely cause too much irritation and can increase the pain rather than relieve it. In addition, electro stimulation and moxibustion are not easily applied along with acupressure.

Acupuncture and acupressure have also been used in conjunction with a prescription for Chinese herbal supplements or teas to enhance pain relief. However, the desired results of pain relief are not immediate, and there is a risk of the herbs conflicting with any other medications the patient is taking and not being prescribed the appropriate dose. Prescription of Chinese herbs should be done by a Chinese medicine practitioner who has extensive training in this field.

An alternative “facelift” has also been conducted where an acupuncturist implements acupuncture or acupressure and follows with a face massage with a cream containing a blend of Chinese herbs. The addition of the massage and herbs increases blood flow and relieves swelling in order to tone the face. However, there is no mention pain relief.

Acupuncture has been used with an herbal compound containing Arnica montana to relieve pain. The method includes injecting the herbal compound in specified acupuncture points, applying a transdermal patch with the herbal compound over specific acupuncture points, and taking orally the herbal compound. Magnetic stimulation has also been used along with the acupuncture and Arnica montana compound. Magnetic stimulation could have negative implications for the patient and involves an additional step for the practitioner.

One method taught for performing acupuncture is inserting a tissue-piercing member into tissue at or adjacent to an acupuncture point and delivering a semi-solid cream, gel, ointment, or lotion. The ingredients of the medicinal compound include the following: anti-atherosclerotic agents, anti-psoriatics, antispasmodics, muscle relaxants, muscle contractants, histamines, antipyretics, analgesics, antihypertensives, anticoagulants, procoagulants, cholesterol-reducing agents, anticonvulsants, cognitive enhancers, cholinergics, anti-cholinergics, anti-Alzheimer's disease agents, sedatives, anti-Parkinson substances, hypnotics, anti-psychotic substances, antacids, antihistamines, antidiabetics, contraceptives, sympathomimetics, coenzymes, adrenergics, adrenergic antagonists, enzyme inhibitors, neurotoxins, neurotransmitters, hormones, antiulcer agents, antiflatulents, proton pump inhibitors, antidiarrheals, antipruritics, anti-emetics, antireflux agents, antiobesity agents, autoimmune disorder agents, anti-cancer substances, immunomodulatory factors, diuretics, anti-glaucoma compounds, anti-inflammatory agents, anti-vertigo medications, local anesthetics, ophthalmics, trophic factors, growth factors, nucleic acids, anti-infectives, vitamins, minerals, nutritional supplements, lubricants, imaging agents, emulsifying stabilizers, herbs, plant extracts, astringents, combinations, derivatives, or precursors thereof. However, the methods described are invasive and likely add to the patient's discomfort.

Another method of pain relief includes placing a small patch with analgesic, particularly capsaicin, on a Chinese medicine acupoint to relieve musculoskeletal pain. However, this method is not used in conjunction with acupuncture or acupressure and could likely have an offensive odor and irritate the skin.

### SUMMARY

What is needed, therefore, is a simple, fast-acting, safe, and minimally-invasive method of enhancing the effects of acupuncture and acupressure while also reducing swelling. Accordingly, the present invention provides a method of applying a topical herbal ointment or cream to the skin to increase vasodilation and circulation and decrease pain and swelling, which may enhance the effects of acupuncture and acupressure. The present invention may mitigate the disadvantages of the aforementioned prior art by reducing additional steps and invasive or irritating procedures.

One general aspect includes a method for aiding acupuncture or acupressure, the method including the steps of identifying an effective application situs for pain on the human body and applying a topical composition onto the effective application situs for pain on the human body. The topical composition may include an effective amount of a combination of herbs or extracts of arnica montana, rhus toxicodendron and aesculus hippocastanum mixed with an effective amount of belladona, in a pharmaceutically acceptable carrier.

Implementations may include one or more of the following features. The method may include examples where the topical composition additionally includes a vasodilator component including one or more of: primrose oil, rosemary, ginger, hamamelis, horse chestnuts, yohimbe, and niacin l-ornithate. The method may include examples where the topical composition includes between about 0.5 to 5% by weight of the vasodilator component. The method may include examples where the topical composition additionally includes one or both of snake venom and snake venom extract. The method may additionally include the step of applying a treatment bandage including a layer of the composition over the effective application situs for pain on human body.

The method where the topical composition additionally includes one or more mobilizers of white blood cell activity including one or more of: lachesis ninta venom, crotalus horridus venom, naja venom, red chinese ginseng, siberian ginseng and korean ginseng, goldenseal, chamomile, and barberry. The method may also include examples where the topical composition includes between about 0.5 to 5% by weight of the one or more mobilizers of white blood cell activity.

One general aspect includes a pain relief bandage. The pain relief bandage may include a sealing layer formable to a contour of an effective application for a situs of pain, a substrate fixedly attached to the sealing layer, and a treating composition on a first surface of the substrate fixedly attached to the sealing layer. The treating composition may include an effective amount of a combination of herbs or extracts of arnica montana, rhus toxicodendron and aesculus hippocastanum mixed with an effective amount of belladona, in a pharmaceutically acceptable carrier.

The details of one or more embodiments of the invention are set forth in the accompanying drawings and the description below. The accompanying drawings that are incorporated in and constitute a part of this specification, illustrate several embodiments of the invention and, together with the description, serve to explain the principles of the invention: Other features, objects, and advantages of the invention will be apparent from the description, drawings, and the claims herein.

### DESCRIPTION OF THE DRAWINGS

The foregoing and other features and advantages of the invention will be apparent from the following, more particular description of preferred embodiments of the invention, as illustrated in the accompanying drawings.

FIG. 1 illustrates an exemplary embodiment of application of a topical homeopathic composition at a site of musculoskeletal pain and at the corresponding pressure point for acupuncture.

FIG. 2 illustrates an exemplary embodiment of application of a topical homeopathic composition at a specified pressure point for fibromyalgia pain.

FIG. 3 illustrates exemplary method steps for applying topical homeopathic composition to the skin in conjunction with application of pressure at a specific pressure point.

FIG. 4 illustrates an exemplary embodiment of a cross section of the skin and possible corresponding placements of a topical homeopathic composition and of an acupuncture needle.

FIG. 5 illustrates an exemplary methods of applying a topical homeopathic composition.

FIG. 6 illustrates an exemplary embodiment of a temporary tattoo placed on the skin at specific energy points.

FIG. 7 illustrates an exemplary treatment bandage.

### DETAILED DESCRIPTION

The present invention relates generally to the method of acupuncture or acupressure that may be used in conjunction with a topical homeopathic composition that may be applied to the skin at the site of an open wound, musculoskeletal pain, or neural pain as well as at a specific pressure point on the body that correlates to that type of pain. In particular, the present invention provides a topical application, such as a cream or gel, which may be applied on or into the skin, wherein the topical application comprises one or more a vasodilator, stimulator of lymphatic activity, or mobilizers. Preferred embodiments include ingredients derived from natural sources, and in some aspects, natural sources processed through homeopathic steps. Homeopathic steps may include, for example, filtration and dilution. In some examples a marking such as a tattoo or a temporary tattoo may be placed on a patient's skin.

More specifically, the components of the composition may comprise a vasodilator, such as, for example, Arnica Montana (a vasodilator, and may reduce swelling at the wound or pain site), primrose oil, rosemary, ginger, Hamamelis, horse chestnuts, Yohimbe, niacin L-ornithate, and the like; a stimulator of lymphatic activity, such as, for example, Echinacea Augustofolia, St. Johns Wort, Belladonna, Devils Claw, Yellow Dock, Burdock, North American Ginseng, Wild Indigo, Pleurisy Root, and Pokeweed; and mobilizers of white blood cell activity, such as, for example, Lachesis Ninta venom, Crotalus horridus venom, Naja venom, Red Chinese Ginseng, Siberian Ginseng and Korean Ginseng, Goldenseal, Chamomile, and Barberry. Each of the components may be used in the amount of about 0.5 to 5%, preferably, 0.5 to 2.0% by weight.

In some aspects, the homeopathic composition may comprise R. toxicodendron, which contains urushiol, a pro inflammatory, and may dull inflammation-associated pain. In some aspects, the homeopathic composition may comprise A. hippocastanum, which contains aescin, a vasoconstrictor and anti-inflammatory, and saponins and surfactants that may reduce swelling and facilitate absorption of ingredients into the dermis.

In some embodiments, the homeopathic composition may comprise snake venom and venom extracts, which have anti-inflammatory capabilities. The snake venom may reduce swellingon or around acupuncture or acupressure treatment points.

In some aspects, the homeopathic composition may comprise prostaglandins, which may have been found to improve nerve conduction velocity. In some embodiments, prostaglandins may be formed to be advantageous to induce prostaglandin production at the site affected by the acupuncture point or area of pain . . . rather than infusing the entire body. Accordingly, the inclusion of 0.5 to 5% by weight of the composition such as primrose oil, flax oil, and crocetin, may be beneficial.

In some embodiments, the homeopathic composition may comprise Caprylyl Glycol, which may function as a humectant and/or stabilizer. Caprylyl glycol may be plant derive or synthetic. Caprylyl glycol may increase antimicrobial activity of other preservatives, and may be used in conjunction with phenoxyethanol and chloroxylenol. The preservative blend may work on the skin's surface to give it a soft and smooth texture.

In some aspects, the homeopathic composition may comprise Caprylic/Capric Triglyceride, which may be produced by chemical reactions between various fatty acids and glycerol (synthetic glycerine). This largely synthetic mixed triester may be derived from coconut oil and glycerin and may comprise an emollient, dispersing agent, and solvent. In some aspects, Caprylic/Capric Triglyceride may comprise spreading properties that may facilitate gliding of the homeopathic composition on the skin.

In some embodiments, the homeopathic composition may comprise Caprylhydroxamic Acid, which may take place of traditional preservatives. Caprylhydroxamic Acid has the best length (C8) of C chain to promote degradation of cell membrane structures. Caprylhydroxamic Acid may maintain a unionized state in acid to neutral and may perform at a neutral pH. Caprylhydroxamic Acid may have excellent efficacy of fungistatic agents.

In some aspects, the homeopathic composition may comprise Centaurea Cyanus Flower Extract or cornflower extract. Centaurea Cyanus Flower Extract may comprise a skin conditioning agent and astringent that is rich in polyphenols, specifically flavonoids and anthocyanins. Cornflower extract may comprise anti-inflammatory properties, which may be effective as a preemptive preparation for an acupuncture treatment.

In some embodiments, the homeopathic composition may comprise Chelidonium Majus or swallow wort, which may commonly be used for treating warts. Swallow wort has appreciable tissue rejuvenating properties. The juice of Chelidonium majus has anti-bacterial/anti-fungal effect, which may be effective to slow or stop excessive bleeding.

In some aspects, the homeopathic composition may comprise Citrus Aurantium Amara (Bitter Orange) Flower Water may comprise flavonoids, which are anti-inflammatory, antibacterial, and antifungal. Orange flower water contains hydrophilic properties and may act as a natural surfactant and emulsifier to enhance the application of skin lotions and other cosmetics. In some aspects, the orange flower water may be safe to use directly on the skin, without requiring dilution, and may be used on blotchy, irritated skin. Orange flower water is a by-product of distillation and may be used in perfumery. Orange flower water is smoothing, soothing, and brightening due to the skin regenerative and vitamin-rich properties of organic citrus flowers.

In some embodiments, the homeopathic composition may comprise Lauric Acid. Lauric Acid is a main fatty acid in coconut oil, which may be used as a moisturizer for the skin and may reduce redness and flaking of skin with minimal adverse side effects.

In some aspects, the homeopathic composition may comprise Citrus aurantium dulcis (orange) peel oil, which may be extracted from the peels of both sweet and sour oranges by cold compression. Orange oil extract may have inflammatory qualities, which may detoxify congested skin and soothe dry or irritated skin, such as may occur in shingles, dermatitis, and acne outbreaks. Orange oil extract may increase the production of collagen and may reduce fine lines and puffiness, which may be effective as a concurring or post acupuncture treatment, particularly around acupuncture points.

In some embodiments, the homeopathic composition may comprise Coenzyme A, which may be adapted from pantothenic acid and adenosine triphosphate. Biologically speaking, this coenzyme plays a vital role in the synthesis and oxidation of fatty acids. Coenzyme A may function as a skin conditioner, emollient, and solvent, and in some aspects, coenzyme A may stimulate collagen production.

In some aspects, the homeopathic composition may comprise Dehydroacetic Acid. As a mild acid, dehydroacetic acid may work with benzyl alcohol as a preservative and antimicrobial to provide a broad protection from contamination. Dehydroacetic acid may function as a fungicide and/or bactericide, which, in some embodiments, may be used in personal care products at a maximum concentration of 0.6%.

In some embodiments, the homeopathic composition may comprise Ginkgo Biloba Leaf Extract, which may be a potent antioxidant that may improve blood flow. Improved blood flow may increase the effectiveness of acupuncture treatment.

In some aspects, the homeopathic composition may comprise Gluconolactone, which is an ester of gluconic acid and is composed of multiple water-attracting hydroxyl groups. Gluconolactone may hydrate skin and enhance the degree of moisturization of the homeopathic composition. Gluconolactone is a polyhydroxy acid (PHA) that is capable of chelating metals and may also function by scavenging free radicals. In some embodiments, gluconolactone may facilitate healing of acupuncture points, and as a gentle ingredient, may be directly applied on sensitive skin.

In some embodiments, the homeopathic composition may comprise Hydrolyzed Lupine Protein, which is a hydrolyzed protein from the seeds of the lupine plant (lupinus). The seeds are high in proteins and contain 35-45% of all the essential amino acids. The hydrolyzed lupine protein may have a restructuring and regenerating effect on skin Lupine peptides are a blend of penta- and hexa-peptides derived from lupin flower protein that may function as an MMP (matrix metalloprotease) inhibitor, which may prevent MMP activity following exposure of the skin to sunlight. Accordingly, the lupine peptides blend may prevent collagen and elastin (the main proteins making up connective tissue) breakdown catalyzed by MMP activity, which may facilitate renewal of the skin's outer layer, which may stimulate the production of collagen & elastin. In some aspects, the hydrolyzed lupine protein may reduce scarring that may be caused by lesions associated with shingles.

In some aspects, the homeopathic composition may comprise Hydroxypropyl tetrahydropyrantriol, which is a sugar-protein hybrid made from xylose, a sugar found abundantly in beech trees. As a series of amino acids, Hydroxypropyl tetrahydropyrantriol is small enough to penetrate the skin, which may allow for deeper and more effective penetration of the homeopathic composition. Hydroxypropyl tetrahydropyrantriol stimulates the production of glycosaminoglycan's (GAGs), or mucopolysaccharides. GAGs, which are an important component of connective tissue and may increase production on an extracellular matrix. In some embodiments, hydroxypropyl tetrahydropyrantriol may facilitate healing of the skin, such as around acupuncture points . . . such as on and around lesions associated with shingles.

A topically applied composition may be in the form of a lotion, cream, gel, or salve. A composition may also contain dimethylsulfoxide or other surfactants, which may allow the active ingredients to penetrate more easily into the dermis and absorb more efficiently into the bloodstream. A synergistic therapeutically effective amount of the combination of a vasodilator, a stimulator of lymphatic activity, and a mobilizer of white blood cell activity that may be topically applied at the site of pain and numbness may be effective to provide relief from the symptoms of a disease causing problems. That is, for use in connection with CRPS diseases such as fibromyalgia, diabetic neuropathy, toxic neuropathy and the like. For example, A. montana, R. toxicodendron, and A. hippocastanum may work synergistically to provide pain relief for fibromyalgia because each of the ingredients may enhance the effects of the other ingredients. A. montana and R. toxicodendron may result in more vasodilation and decreased pain when used together in homeopathic dilutions rather than each ingredient on its own.

Furthermore, a topical homeopathic composition may also contain essentials oils such as tea tree oil that have natural antibacterial properties. A topical homeopathic composition may also contain aspirin which may aid in pain reduction and increased blood flow at the body's pressure points. Essential oils and traditional analgesics such as asprin may enhance vasodilation and pain relief. Furthermore, essential oils and analgesics may synergistically enhance the effects of a homeopathic topical remedy as well as prevent any dermal infections that may occur from needle insertions or wound exposure.

Snake venom and venom extracts have been found to stimulate the production of both pro- and anti-inflammatory cytokines. The pro-inflammatory cytokines are produced which may lead to an increase in a localized heat sensation. Anti-inflammatory cytokines are produced which may help in eliminating pain and edema. Lachesis muta snake venom may produce a hemorrhaging effect that may increase blood flow and circulation in the area where a topical homeopathic composition may be applied. Crotalus horridus snake venom may produce anti hemorrhagic effects that may decrease inflammation and blood flow in an area where a topical homeopathic composition may be applied. Snake venom from species of Naja may induce edema and a tingly sensation that may dull pain in an area where a topical homeopathic composition may be applied. For example, a topical homeopathic cream containing snake venom may be applied to a wound site which may or may not be at a corresponding acupuncture or acupressure point. In some embodiments, a topical homeopathic cream with snake venom may make a wound site more inflamed for a few minutes. Another effect of a topical homeopathic cream with snake venom may be where a wound site that may be irritated and inflamed may become less painful and less swollen and red. A wound may be on or below the skin.

In some examples, a topical homeopathic composition may comprise an ingredient capable of promoting penetration and absorption of the ingredients into the body. A topical homeopathic composition may comprise ingredients which may include large molecules that are not readily absorbed into the skin. Therefore, a component of a carrier surfactant, emollient, or gel may facilitate pinocytosis, so a large-molecule ingredient may penetrate through the skin and be absorbed into the bloodstream. As a result, vasodilation and opening of energy channels may occur.

In some aspects, a topical homeopathic composition may be applied in regions of the body where penetration and/or absorption may be more efficient, such as parts of the body with larger surface area like the chest or back. In some aspects, particularly in areas of the body where penetration may be difficult, such as the knees or elbows, an additive or a preparation step may increase penetration. For example, an area may be prepared by a simple alcohol swab which may remove surface contaminants that may inhibit penetration.

Another example may be exfoliation before applying a topical homeopathic composition. In some aspects, a topical homeopathic composition may also include particles that may contain exfoliate properties. Exfoliates may be physical particles that may be botanical such as ground seeds or synthetic such as microbeads. Exfoliates may also be molecular such as glycolic acid. In such aspects, an exfoliate in a topical homeopathic composition may remove skin from the stratum corneum which may improve penetration of ingredients into the dermis and may improve absorption of ingredients into the bloodstream. An example of a use is when a homeopathic composition with an exfoliate may be applied to an acupressure point and rubbed in for several minute. A homeopathic composition with an exfoliate may be wiped off while still may be producing pain relief and circulatory effects at an acupressure point.

In some aspects, a topical homeopathic composition may reduce the risk of electric shock, burns, allergic reactions, and dermatitis, which may occur from other acupuncture enhancements such as electro stimulation and moxibustion. In some embodiments, a topical homeopathic composition may be applied to the skin before, during, and/or after insertion of acupuncture needles or application of pressure. A topical homeopathic composition may boost circulation and open energy channels to promote healing in conjunction with a needle or pressure. Those schooled in the art will understand that electrostimulation and moxibustion, may also be used in conjunction with acupuncture or acupressure and may produce additional enhancing effects. A homeopathic composition may or may not be used with acupuncture or acupressure and an enhancement such as moxibustion.

Overall, an application of a topical homeopathic composition may increase blood flow to a wound to promote healing and may inhibit pro-inflammatory cytokines. In some aspects, a topical homeopathic composition may decrease swelling to reduce edema associated pain.

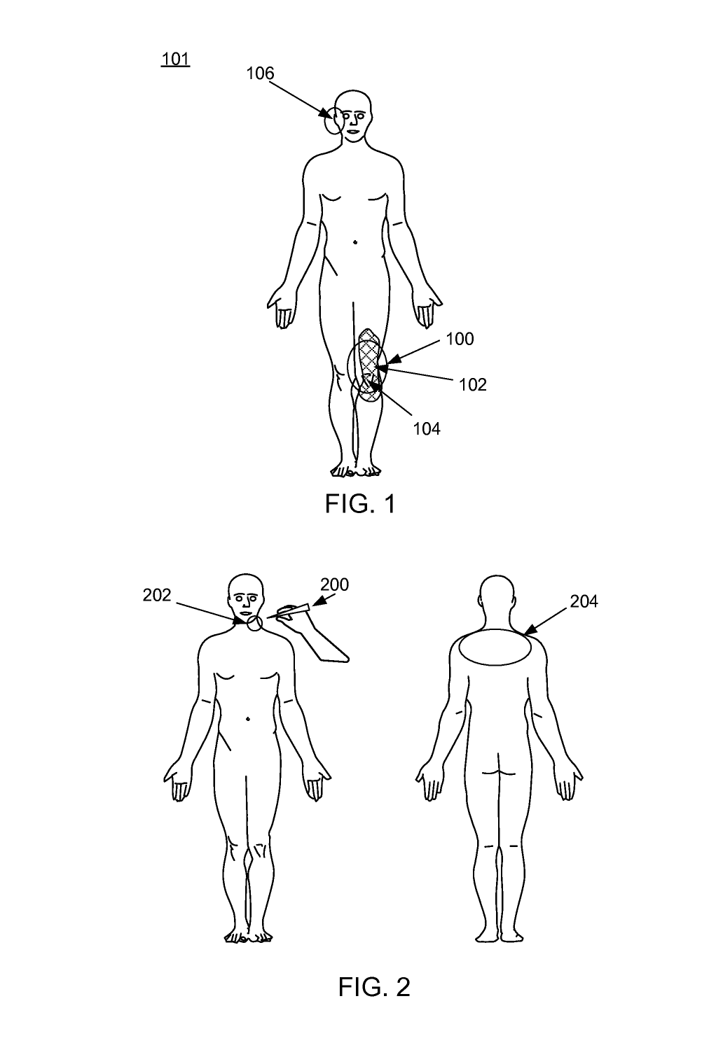

Referring to FIG. 1, an exemplary embodiment of application of a topical homeopathic composition at a site of musculoskeletal pain and at the corresponding pressure point for acupuncture is illustrated. In some embodiments, a person may apply a topical homeopathic composition 102 to an area of pain and/or inflammation 100. An application of a topical homeopathic composition 102 may be coupled with acupuncture or acupressure at or near area of inflammation 100.

In some aspects, a topical homeopathic composition 102 may be applied to an area of pain and/or inflammation 100, which may be different from a pressure and/or acupuncture site 106 to further enhance pain relief. For example, a acupressure or acupuncture point site 106, for pain relief of a knee joint 104 may be located on an on an ear of the patient 101. In some such examples, a topical composition 102 may be applied to the area of pain and/or inflammation 100, (such as a knee joint area) before, during, or after an acupressure or acupuncture procedure on an acupressure or acupuncture site 106 on the ear.

In other aspects, an affected pain area may be separate from one or both the acupuncture point and the topical application area. For example, as a treatment for a headache, an acupuncture point may be located on the base of the neck, and a topical composition may be applied to a region of the hand. Simultaneous therapy of acupuncture and topical application may provide increased and/or longer lasting pain management. Separate sites for acupuncture points and topical application may reduce risk of contamination of the acupuncture needle.

Referring to FIG. 2, an exemplary embodiment of application of a topical homeopathic composition at a specified pressure point for fibromyalgia pain is illustrated. In some embodiments, such as for fibromyalgia, an acupressure or acupuncture point 202 may be located on the neck, and a topical composition may be applied to the upper back 204. A needle or finger pressure 200 may be applied at an acupuncture or acupressure pressure point 202. In some embodiments, an acupuncture point 202 and the location of pain (not indicated) may be in the same area wherein a topical homeopathic composition may be applied before, during, or after placement of a needle or pressure to relieve nerve pain.

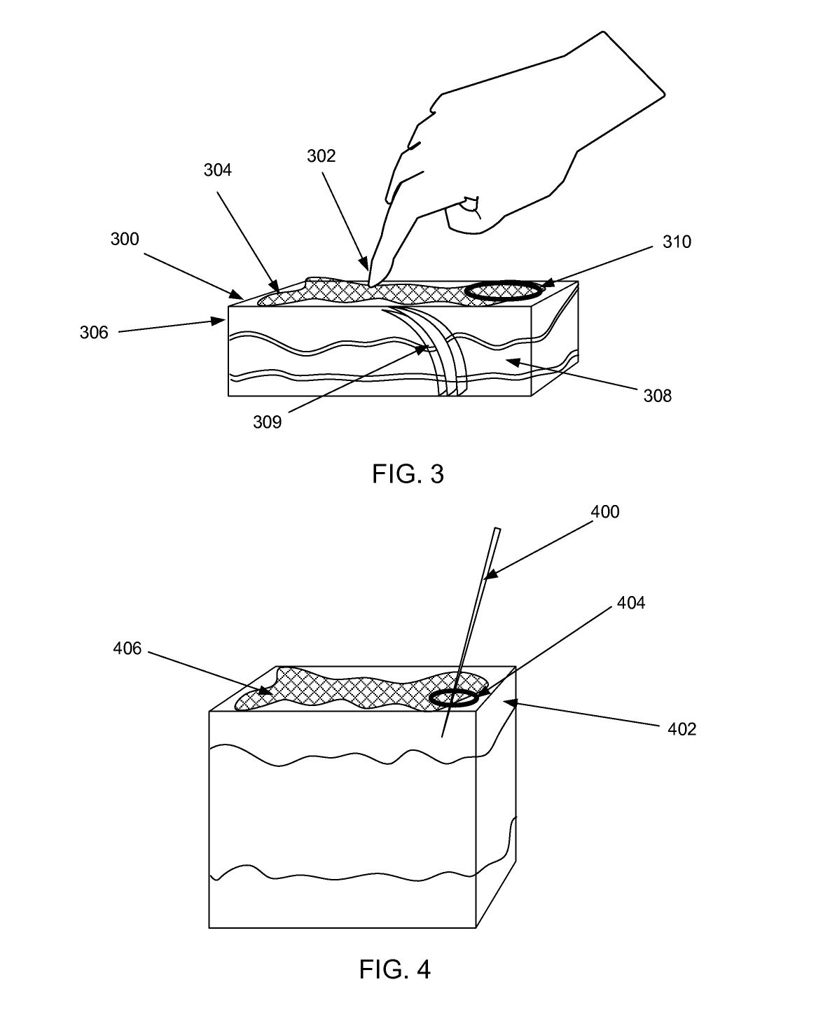

Referring to FIG. 3, exemplary embodiments for applying a topical homeopathic composition to the skin in conjunction with application of pressure at a specific pressure point is illustrated. In some embodiments, a finger 302 may be pressed onto the skin 300 of a person in the same area where a topical homeopathic composition 304 was, is, or may be applied. The pressure may reach the dermis 306. The pressure from the finger 302 along with the topical homeopathic composition 304 may increase energy flow and vasodilation of blood vessels 310 in the dermis 306. The vasodilation 310 may release tension in muscles 308 and/or nerve 309 inflammation. Therefore, in such embodiments, a topical homeopathic composition 304 along with pressure from a finger 302 may enhance and expedite pain relief.

Referring to FIG. 4, an exemplary embodiment of a cross section of the skin and possible corresponding placements of a topical homeopathic composition and of an acupuncture needle is illustrated. In some embodiments, an acupuncture needle 400 may be inserted into the dermis 402 where target blood vessels and energy channels may be located that correspond to a person's pain and/or inflammation. In some embodiments, a topical homeopathic composition 406 may be applied on the epidermis at an acupuncture and/or acupressure site 404 and may absorb into the dermis to further result in vasodilation and energy flow. In some embodiments, an acupuncture needle 400 coupled with a topical homeopathic composition 406 may target nerves and may relieve nerve pain.

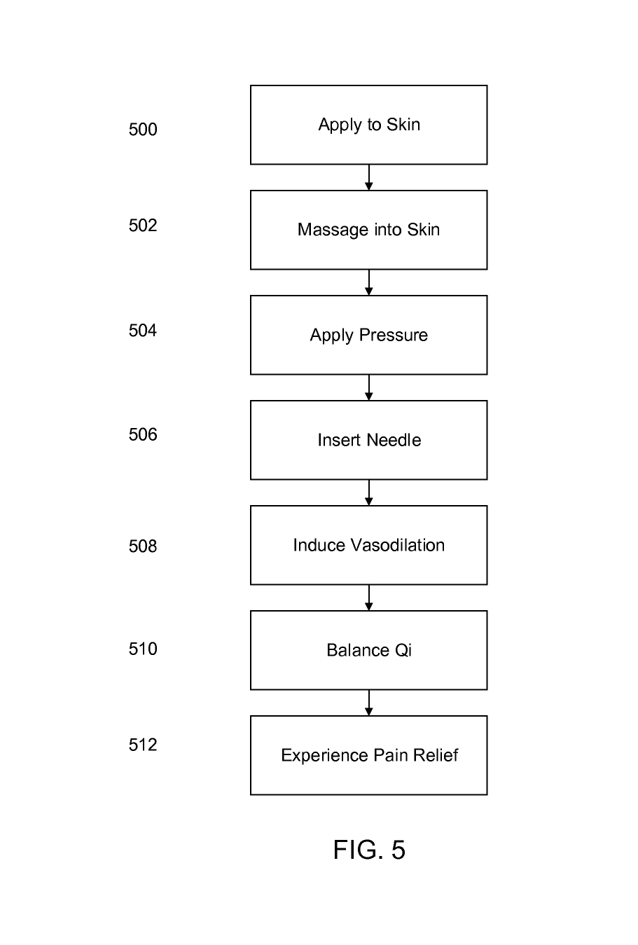

Referring to FIG. 5, exemplary method steps of applying a topical homeopathic composition is illustrated. In some methods, a person may apply a topical homeopathic composition to the skin 500. In some methods, a person may massage a topical homeopathic composition into the skin 502. In some methods, a person may implement pressure with the fingertips 504. In some methods, a person may insert a needle into an acupuncture point 506. In some methods, vasodilation may be induced 508. In some methods, Qi or energy flow may be balanced 510. In some methods, a person may experience pain relief 512.

In some aspects, a topical homeopathic composition may further increase blood flow and open the body's energy channels, which may provide a synergistic enhancement to acupuncture and acupressure treatments. A topical homeopathic composition applied in conjunction with acupuncture or acupressure may reduce the number and duration of acupuncture or acupressure treatments traditionally needed to treat an ailment.

In some aspects, an acupuncturist or acupressurist may apply a topical homeopathic composition to the skin before, during, or after a treatment session. In some aspects, a composition may be applied for up to thirty minutes before acupuncture or acupressure treatment begins. A composition may have therapeutic effects for up to four hours without needing reapplication. A composition may be rubbed into the skin or simply spread on the skin. In some aspects, patient person may continue to apply a topical homeopathic composition for a period following an acupuncture treatment, which may reduce the number of acupuncture or acupressure sessions needed to eliminate or significantly reduce pain. A composition may reduce pain and swelling as soon as 1 minute after topical application.

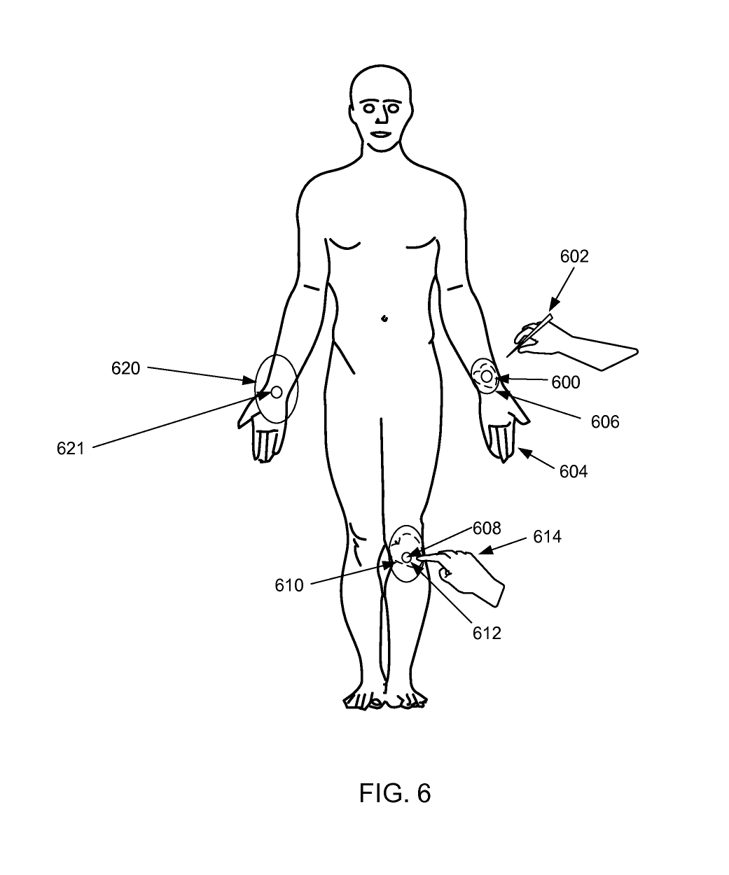

Referring to FIG. 6, in some embodiments, a marking may be placed upon a patients' body to indicate one or more of: where an acupressure and an acupuncture point is located on a patient's body and where a topical composition to relieve pain and vasodilate an area may be applied in conjunction with the acupressure and acupuncture points are located. In some exemplary embodiments, a marking may include a permanent or a temporary tattoo placed on the skin at specific energy points, as illustrated. In some embodiments, a temporary tattoo 600 may be placed on a small, specific pressure or acupuncture point on the body such as the wrist. A marking 600 such as a temporary or permanent tattoo may include a central spot 604 that may indicate the exact pressure point or acupuncture point. An acupuncture needle 602 may be placed on the central point. The marking 600 may indicate a circumference 606 that may designate a preferred placement of a homeopathic topical composition.

In some aspects, a marking 600 such as the temporary or permanent tattoo may include instructions for professionals or for at-home DIY use. Instructions may include a type and frequency of treatment and related areas that may also be treated in conjunction with the marked area. A temporary tattoo may be included in a set or kit that may include various sizes of temporary tattoos. A set or kit of temporary tattoos may be color coded to indicate a type of use, a type of placement, and/or whether a temporary tattoo may be used for a specific pressure or energy point or whether a temporary tattoo may be used for a general area where pressure or acupuncture may be applied anywhere within a temporary tattoo's indicated circumferences. Various sizes of temporary tattoos may differ based on a disease being treated, some symptoms a person may experience, or the size of a wound.

In some embodiments, a temporary tattoo may be placed on a larger, more general acupuncture or acupressure point that is distal from an actual wound site or pain site. A marking 600 such as the temporary tattoo may include a shape 610 indicating a general area where pressure may be applied. Pressure may be applied anywhere within a shape 610 or on an exact pressure point 608. A needle, or pressure from a finger or instrument 614 may be applied on an exact central pressure point 608. An outer circumference 612 may indicate a preferred placement of an application of a topical composition. An outer circumference 612 may also indicate a placement or a treatment area for any acupuncture or acupressure enhancements, such as moxibustion or electrostimulation, other than a topical composition. A topical composition or other enhancement may be placed in between tattoo marks 610 and 612, within line type tattoo mark 610 and 612, or over an entire area a temporary tattoo covers. A topical composition may also be applied outside of a temporary tattoo's boundaries.

In some aspects, a temporary tattoo may be applied before, during, or after application of acupressure, acupuncture, or application of a topical composition. A temporary tattoo may contain a water-soluble composition or may contain a lipo-soluble composition. A temporary tattoo may be placed on an open wound, directly on a specific site for pressure or acupuncture point 621, or in a general pain location 620. A temporary tattoo may include soothing botanicals or analgesics that may aid in and may prevent further pain and/or infection. A temporary tattoo may be applied with water or an emollient. A temporary tattoo may be wiped off immediately after use or may last a longer time period such as five days. A temporary tattoo may be used for a single treatment application or may be applied once and used for an entire duration of an acupuncture or acupressure treatment course.

In some embodiments, a temporary tattoo may change colors, which may indicate severity of pain or blockage of an energy channel where a temporary tattoo is placed. A color changing temporary tattoo may be activated by body heat, which may indicate energy channel blockage, pain, inflammation, or edema. In some aspects, a color changing temporary tattoo may have a color meter ranging from black, which may signify no energy channel blockage, to blue, which may signify moderate energy channel blockage, to red, which may signify severe energy channel blockage.

For example, a temporary tattoo may have a default color of black when a temporary tattoo is placed on the skin. A temporary tattoo may change to a red color at a central point, which may indicate severe energy channel blockage, edema, inflammation, or pain. A temporary tattoo may change to a color blue on an outer circumference, which may indicate a moderate energy channel blockage, edema, inflammation, or pain. A color change in a temporary tattoo may aid a person to more accurately target a most severe energy channel blockage, which may aid in more effective acupuncture or acupressure treatment.

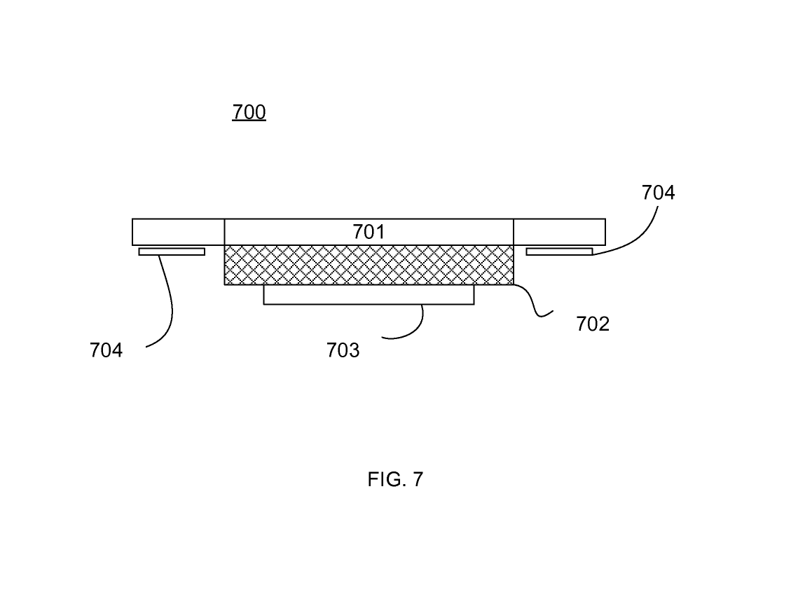

Referring now to FIG. 7, in some embodiments, a treatment bandage 700 may include a substrate 702 dosed with a homeopathic composition 703. The substrate 702 may be comprised of a porous portion impregnated with homeopathic composition 703. The substrate 702 may therefore include one or both of a natural fiber, such as a cotton or other plant based fiber and a synthetic fiber or gel. The substrate 702 may also include a dissolvable starch that may dissolves into the skin. The substrate 702 may be fixedly attached to a sealing layer 701 that generally provides a boundary between the homeopathic composition 703 and an ambient environment. The sealing layer 701 may include a polymer, vinyl, latex, plastic or other material formable to a contour of a human body part.

In some embodiments, the sealing layer may include an adhesive layer 704 for removeably attaching the substrate to a body part in a fashion that allows the homeopathic composition 703 to be brought into contact with the skin of a patient and maintained in contact with the skin. The sealing layer allows that homeopathic composition 703 to remain relatively undisturbed by an ambient environment.

### ILLUSTRATIVE EXAMPLES

Illustrative examples of homeopathic composition formulas are listed and described below. These are exemplary only and should not be considered as limiting.

### Example 1

A homeopathic gel prepared by admixing the following ingredients.

| Ingredient | Wt. % |
|---|---|
| Carbomer 940 | 2.10 |
| Xantham gum | 0.15 |
| Propylene glycol | 51.94 |
| Dipropylene glycol | 10.00 |
| Ethoxydiglycol | 15.00 |
| Dimethylisosorbide | 10.00 |
| Aloe Vera gel | 2.00 |
| Surfactant | 0.05 |
| Amica Montana | 2.50 |
| Belladooa | 2.50 |
| Rhus toxicodendron | 2.00 |
| Aescuius hippocastanum | 1.76 |

Although the specific activity of each of either plants or herbs have been recognized, it has been surprisingly found that the combination as now claimed has been found to produce the desired effect. The composition is applied to the limb having pain 1-8 times per day.

### Example 2

A creme was formed by admixing the following ingredients:

| Ingredient | | |
|---|---|---|
| Aescuius hippocastanum extract | 6 | mcg/g |
| Amica Montana extract | 6 | mcg/g |
| L-arginine | 6 | mcg/g |
| Echinacea augustfolia extract | 6 | mcg/g |
| Rhus toxicondendron extract | 6 | mcglg |
| Ruta graveoleus extract | 6 | mcg/g |
| Graphites | 6 | mcg/g |
| Crotalus horridus extract | 0.08 | mcg/g |
| Heloderma horridum extract | 0.08 | mcg/g |
| Lachesis extract | 0.08 | mcg/g |
| Naja extract | 0.08 | mcg/g |
| Isopropyl myristate | 30 | mg/g |
| Brij 72 | 2 | mg/g |
| Brij 72IS | 2 | mg/g |
| Water | 83 | mg/g |
| Methyl paraben | 0.2 | mg/g |
| Propyl paraben | 20 | mcg/g |
| Glycerine | 3 | mg/g |
| 20% NaOH | 3 | mg/g |
| Dowcil 200 | 50 | mcg/g |
| Akodel 112 | 2 | mg/g |

The creme may be used to treat cracked skin on feet or anhidrosis, which is associated with CRPS.

### Example 3

A lotion is prepared by admixing the following ingredients:

| Ingredient | Wt. % |
|---|---|
| Ginger | 1.10 |
| Propylene Glycol Stearate | 6.50 |
| Isocetyl alcohol | 5.00 |
| PEG-100 Stearate | 1.20 |
| Water | 69.90 |
| Echinacea augustfolia extract | 3.00 |
| Methyl paraben | 0.20 |
| Propylene glycol | 12.00 |
| Sorbitan palmitate | 0.60 |
| Arnica Montana | 3.00 |
| Aesculus hippocastanum extract | 2.00 |
| Barberry | 1.00 |
| Mate extract | 0.50 |

If desired, 3% by weight capsaicin can be added. The lotion can be used to treat a patient suffering from fibromyalgia.

### Example 4

### Preparation of a Gel

| Ingredient | Wt. % |
|---|---|
| Arnica Montana | 5.00 |
| Primrose oil | 3.00 |
| Arginine base (10% solution) | 5.00 |
| (Ajinomoto) | |
| Carbopol 940 | 0.40 |
| Butylene glycol | 6.50 |
| Echinacea augustfolia | 3.00 |
| Chamomile glycolic extract | 3.00 |
| Crotalus horridus venom | 0.50 |
| Preservative | 0.10 |
| Fragrance | 0.10 |
| Deionized water | g.s. |

To 20 ml of water with stirring is added the Carbopol 940. The mixture is stirred until hydration is complete and then butylene glycol is added. The arginine base is then added to the mixture. The remaining ingredients are mixed together and added to the first mixture. The mixing is continued until uniform. The composition can be applied 1-8 times daily to reduce the pain resulting from CRPS.

### Example 5

A gel is prepared by admixing the following ingredients:

| Ingredient | Wt. % |
|---|---|
| Propylene Glycol | 43 |
| Polyacrylic acid | 2.1 |
| Dipropylene Glycol | 16 |
| Xantbam Gum | 0.15 |
| Etboxydiglycol | 15 |
| Dimetbylisosorbide | 10 |
| Ascorbic Acid | 2 |
| Chloroxylenol | 0.2 |
| Linoleamidopropyl PG- | 1.5 |
| diammonium chloride phosphate | |
| Glyceretb 4.5 Lactate | 2 |
| Naja venom extract | 2 |
| Echinacea augustfolia | 2 |
| Octoxynol-9 | 0.5 |
| Primrose Oil | 2 |
| Cocamidopropyl PG-dimon | 1 |
| chloride phosphate | |
| Water | 6 |
| Ginger | 0.44 |

Ingredients 1 and 2 are mixed to disperse and form a gel. About 80% of ingredient 3 is mixed with ingredient 4, added to the gel slightly heated with admixture. The balance of 3 is mixed with ingredients 5-17 and added to the gel at 38 degrees. After mixing, the pH is adjusted to about 4 and then the gel is brought to room temperature. Arnica Montana may also be added to have a plurality of dual acting ingredients. Capsaicin together with the ginger can be used to relieve pain.

### CONCLUSION

A number of embodiments of the present invention have been described. While this specification contains many specific implementation details, there should not be construed as limitations on the scope of any inventions or of what may be claimed, but rather as descriptions of features specific to particular embodiments of the present invention.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

While the invention has been described in conjunction with specific embodiments, it is evident that many alternatives, modifications and variations will be apparent to those skilled in the art in light of the foregoing description. Accordingly, this description is intended to embrace all such alternatives, modifications, and variations as fall within its spirit and scope.

Although shown and described in what is believed to be the most practical and preferred embodiments, it may be apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims

What is claimed is:

1. A method for enhancing benefits of one or both of acupuncture or acupressure treatment, the method comprising the steps of:
identifying a situs of pain on a human body;
identifying at least one of: an acupuncture and an acupressure point on the human body associated with treating the situs of pain; and
applying a topical composition onto one or both the situs of pain and the acupuncture and acupressure point on the human body associated with treating the situs of pain, wherein the composition comprises a vasodilator component and a combination of extracts of Arnica Montana, Rhus toxicodendron and Aesculus hippocastanum mixed with belladona, in a pharmaceutically acceptable carrier.

2. The method of claim 1 additionally comprising the steps of placing a marking on a patient's skin indicating one or more of: a specific acupressure or acupuncture point on the human body.

3. The method of claim 2 wherein the marking comprises one or both of: a Tattoo and a Temporary tattoo.

4. The method of claim 2 additionally comprising the step of indicating with the marking a central spot that may indicate one or both of: a pressure point or acupuncture point.

5. The method of claim 4 additionally comprising the step of indicating with the marking an area that may designate a preferred placement of the topical composition.

6. The method of claim 1 wherein the vasodilator component comprises one or more of: primrose oil, rosemary, ginger, Hamamelis, horse chestnuts, Yohimbe, and niacin L-ornithate and the topical composition comprises between about 0.5 to 5% by weight of the vasodilator component.

7. The method of claim 6 wherein the topical composition additionally comprises a component comprising a stimulator of lymphatic activity comprising one or more of: Echinacea Augustofolia, St. Johns Wort, Belladonna, Devils Claw, Yellow Dock, Burdock, North American Ginseng, Wild Indigo, Pleurisy Root, and Pokeweed.

8. The method of claim 7 wherein the topical composition comprises between about 0.5 to 5% by weight of the component comprising the stimulator of lymphatic activity.

9. The method of claim 7 wherein the topical composition additionally comprises one or more mobilizers of white blood cell activity comprising one or more of: mobilizers of white blood cell activity, such as, for example, Lachesis Ninta venom, Crotalus horridus venom, Naja venom, Red Chinese Ginseng, Siberian Ginseng and Korean Ginseng, Goldenseal, Chamomile, and Barberry.

10. The method of claim 9 wherein the topical composition comprises between about 0.5 to 5% by weight of the one or more mobilizers of white blood cell activity.

11. The method of claim 7 wherein the topical composition additionally comprises a humectant comprising Caprylyl glycol in conjunction with phenoxyethanol and chloroxylenol.

12. The method of claim 11 wherein the topical composition comprises between about 0.5 to 5% by weight of the humectant.

13. The method of claim 7 wherein the topical composition additionally comprises a preservative comprising Caprylhydroxamic Acid.

14. The method of claim 7 wherein the topical composition additionally comprises one or more flavonoids, which are anti-inflammatory, antibacterial, and antifungal.

15. The method of claim 7 wherein the topical composition additionally comprises one or both of snake venom and snake venom extract.

16. The method claim 15 wherein the snake venom comprises a Lachesis muta snake venom.

17. The method claim 15 wherein the snake venom comprises a Crotalus horridus snake venom.

18. The method of claim 17 wherein the composition comprises a gel.

19. The method of claim 17 wherein the composition comprises a lotion.

20. The method of claim 17 wherein the composition comprises a cream.
